(19) United States
(12) Patent Application Publication
(10) Pub. No.: US 2024/0277759 A1
(43) Pub. Date: Aug. 22, 2024

(54) ANTIBACTERIAL COMPOSITION COMPRISING TRANSITION METAL DICHALCOGENIDE AS AN ACTIVE INGREDIENT

(71) Applicant: INDUSTRY-UNIVERSITY COOPERATION FOUNDATION HANYANG UNIVERSITY ERICA CAMPUS, Gyeonggi-do (KR)

(72) Inventors: Jong-Ho Kim, Gyeonggi-do (KR); Yoonhee So, Gyeonggi-do (KR)

(21) Appl. No.: 18/569,116

(22) PCT Filed: May 20, 2022
(86) PCT No.: PCT/KR2022/007210

§ 371 Date: Dec. 11, 2023

(30) Foreign Application Priority Data
Jun. 17, 2021 (KR) 10-2021-0078892
Apr. 19, 2022 (KR) 10-2022-0047871

(51) Int. Cl. A61K 33/24; A61K 9/70; A61K 47/32; A61P 31/04

(52) U.S. Cl. CPC A61K 33/24; A61K 9/7007; A61K 47/32; A61P 31/04

(57) ABSTRACT

Provided is an antibacterial composition containing transition metal dichalcogenide (TMD) as an active ingredient and a method comprising: adding bulk TMD to a polyacrylic acid (PAA) solution to prepare a mixed solution; ultrasonically treating the mixed solution; centrifuging the ultrasonically treated mixed solution to obtain a precipitate from a supernatant; and redispersing the precipitate in water and centrifuging the same to obtain a PAA-TMD nanosheet from the supernatant.

### TECHNICAL FIELD

The present invention relates to an antibacterial composition containing transition metal dichalcogenide (TMD) as an active ingredient.

### BACKGROUND ART

Diseases caused by bacterial infections are one of the world's major public health problems, and the development of antibacterial agents and antibiotics, especially against multidrug resistant bacteria, is considered very important.

As multi-drug resistant bacteria are generated and threaten the development of traditional drugs, transition metal ions and metal nanoparticles including Cu, Zn, Co, and Ag are attracting attention as substitutes for new antibacterial agents that affect bacterial cells. Metal nanoparticles exhibit excellent antibacterial activity due to their unique properties such as nanometer effect and high surface area/volume ratio. However, excessive metal ions that have reached the form of nanoparticles cause problems in normal tissues as well as bacteria.

Therefore, it is necessary to develop a new antibacterial material capable of killing pathogenic bacteria such as Staphylococcus aureus, Bacillus cereus, epiphytophthora bacteria, and Bacillus oryzae at a low concentration that does not affect normal cells.

### DISCLOSURE OF THE INVENTION

### Technical Problem

Therefore, the present invention has been made in an effort to provide a novel metal-based antibacterial composition capable of killing pathogenic bacteria such as Staphylococcus aureus, Bacillus cereus, epiphytophthorus, and Bacillus archaeus at a low concentration.

### Technical Solution

In order to solve the above problem, the present invention provides an antibacterial composition comprising transition metal dichalcogenide (TMD) as an active ingredient.

In an embodiment of the present invention, the transition metal dichalcogenide (TMD) is a nanosheet.

The transition metal dichalcogenide may include at least one selected from the group consisting of WS₂, MoS₂, WSe₂, and MoSe₂.

In an embodiment of the present invention, the transition metal dichalcogenide (TMD) nanosheet is polyacrylic acid (PAA) functionalized by the transition metal dichalcogenide.

In an embodiment of the present invention, the dichalcogenide of the transition metal dichalcogenide (TMD) is Se.

The present invention relates to a method for preparing an antibacterial composition, and provides a method for preparing an antibacterial composition, comprising the steps of: preparing a mixed solution by adding bulk TMD to a polyacrylic acid (PAA) solution; ultrasonically treating the mixed solution; obtaining a precipitate from a supernatant by centrifuging the ultrasonically treated mixed solution; and obtaining an PAA-TMD nanosheet from the supernatant by redispersing the precipitate in water and centrifuging the same.

The transition metal dichalcogenide may include at least one selected from the group consisting of WS₂, MoS₂, WSe₂, and MoSe₂.

### Advantageous Effects

The present invention can kill pathogenic bacteria such as Staphylococcus aureus, Bacillus cereus, Epidermococcus aureus, and Bacillus subtilis when the nanosheet of Transition Metal Dicalcogenide (TMD) is used at a very low concentration. Further, the functional TMD nanosheet of the present invention has a novel effect on both multidrug-resistant gram-positive and negative bacteria as an excellent antibacterial activity.

Therefore, the TMD nanosheet according to the present invention kills pathogenic bacteria, and shows excellent antibacterial activity against multi-drug resistant bacteria, and thus can be used as a material for preventing and treating diseases caused by pathogenic bacteria and multi-drug resistant bacteria.

### BRIEF DESCRIPTION OF THE DRAWINGS

FIG. 1 is a TEM image of a PAA-TMD nanosheet prepared according to an embodiment of the present invention (PAA-WS₂, PAA-WSe₂, and PAA-MoSe₂ from the left, and a scale bar is 20 nm).

FIG. 2 shows the results of analysis of side size and height profiles of PAA-TMD nanosheets (PAA-WS₂, PAA-WSe₂, and PAA-MoSe₂ from the left).

FIG. 3 is a Raman spectrum analysis result of PAA-TMD nanosheets, and FIG. 4 is a FT-IR analysis result.

FIG. 5 shows the results of analysis of the relative survival rates of Staphylococcus aureus (S. aureus), Bacillus cereus (B. cereus), Escherichia coli (E. coli), and Salmonella typhimurium (S. typhimurium) treated with PAA-TMD nanosheets at a concentration of 20 g/mL.

FIG. 6 shows the results of concentration-dependent antibacterial activity analysis of PAA-WSe₂ and PAA-MoSe₂ against Staphylococcus aureus (S. aureus).

FIG. 7 is an analysis result of the antibacterial activity of PAA-WSe₂ against Gram-positive bacteria.

FIG. 8 shows the results of analyzing the viability of multidrug-resistant Staphylococcus aureus (S. aureus) and Escherichia coli (E. coli) treated with PAA-WSe₂ or PAA-MoSe₂.

FIG. 9 is pictures of Staphylococcus aureus (S. aureus), Escherichia coli (E. coli), Bacillus cereus (B. cereus) and Salmonella typhimurium (S. typhimurium) colonies grown on nutrient agar plates after treatment with various concentrations of PAA or PAA-TMD nanosheets for 6 hours.

FIG. 10 is a picture of multidrug resistant Staphylococcus aureus (S. aureus) and Escherichia coli (E. coli) colonies grown on nutrient agar plates after treatment with various concentrations of PAA-TMD nanosheets.

FIG. 11 is a scanning electron microscope (SEM) image of a)-b) Staphylococcus aureus (S. aureus), c)-d) Escherichia coli (E. coli), and shows results before and after treatment with PAA-WSe₂ (20 μg/mL) for 3 hours (scale bar: 1 m).

### BEST MODE FOR CARRYING OUT THE INVENTION

Hereinafter, a preferred embodiment of the present invention will be described in detail with reference to the accompanying drawings.

Before describing the present invention in detail, the terms or words used in the present specification should not be unconditionally construed as a general or dictionary meaning, and the inventors of the present invention may appropriately define and use the concepts of various terms in order to describe their invention in the best way.

Furthermore, it should be noted that these terms or words should be interpreted as meanings and concepts consistent with the technical idea of the present invention.

That is, the terms used in the present specification are merely used to describe the preferred embodiments of the present invention, and are not used to specifically limit the contents of the present invention.

It should be noted that these terms are defined in consideration of various possibilities of the present invention.

In addition, in the present specification, a singular expression may include a plurality of expressions unless the context clearly indicates a different meaning.

In addition, it should be noted that even when expressed in plural, it may include a singular meaning.

When a feature element is described as “including” another feature element throughout the specification, it may mean that any other feature element may be further included without excluding any other feature element unless there is a description of a particular opposite meaning.

In order to solve the above-described problem, the present invention utilizes a Transition Metal Dicalcogenide (TMD) material. The transition metal dichalcogenide has a structural formula of MX2 (M=transition metal, X=chalcogen element), the structure of the transition metal dichalcogenide is a layered structure similar to graphene, the interval between layers is about 6-7 Å, and it is formed by a strong in-plane covalent bond and a weak out-of-plane van der Waals force. Transition metals of transition metal dichalcogenides are tungsten (W) and molybdenum (Mo), and chalcogen elements include sulfur (S), selenide (Se), telluride (Te), and the like.

The present invention can kill pathogenic bacteria such as Staphylococcus aureus, Bacillus cereus, Epidermococcus aureus, and Bacillus subtilis when the nanosheet of Transition Metal Dicalcogenide (TMD) is used at a very low concentration. Further, the functional TMD nanosheet of the present invention has a novel effect on both multidrug-resistant gram-positive and negative bacteria as an excellent antibacterial activity.

Therefore, the MD nanosheets according to the present invention kill pathogenic bacteria and exhibit excellent antibacterial activity against multidrug resistant bacteria, and thus can be used as a material for preventing and treating diseases caused by pathogenic bacteria and multidrug resistant bacteria.

Hereinafter, the present invention will be described in more detail according to the drawings and experimental examples. However, the scope of the present invention is not limited according to the following experimental examples.

### Example

### TMD Nanosheet Synthesis

Bulk TMD (WS₂ 0.6 g, WSe₂ 0.83 g, MoSe₂ 0.61 g) was added to 20 mL of polyacrylic acid (PAA) solution (2 mg/mL). The mixture was then sonicated for 5 hours (pulse-on for 6 seconds and pulse-off for 2 seconds).

The resulting solution was centrifuged at 1,977×g for 1 hour to generate a supernatant. Then, the supernatant was centrifuged at 15,344×g for 1.5 hours to obtain a precipitate, which was redispersed in 8.5 mL of water. Then, the solution was centrifuged at 3024×g for 1.5 hours, and the supernatant was finally collected to obtain a PAA-TMD nanosheet which is a TMD nanosheet according to the present invention.

### Experimental Example

### TMD Analysis

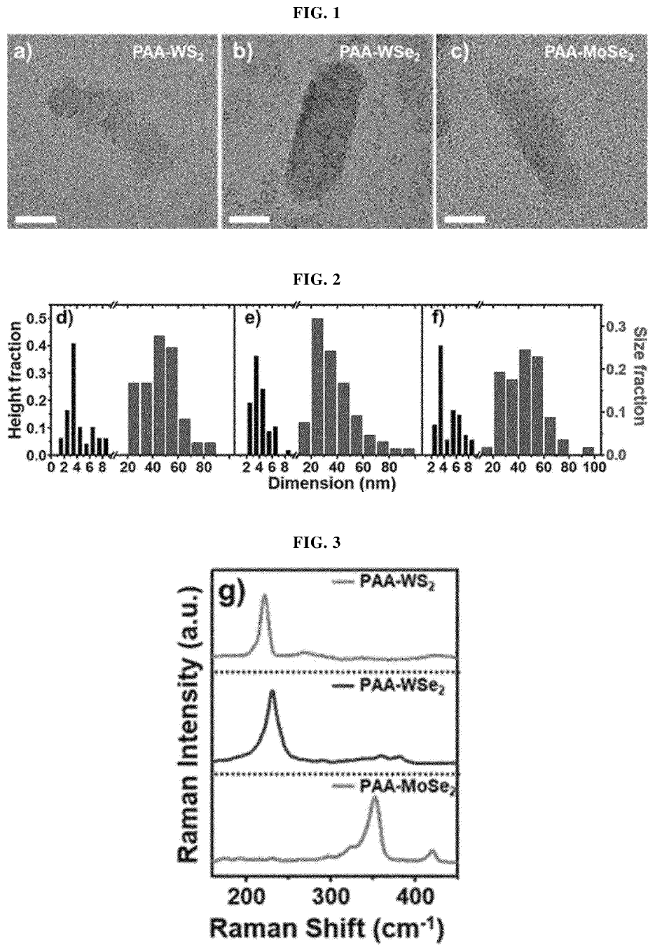

FIG. 1 is a TEM image of a PAA-TMD nanosheet prepared according to an embodiment of the present invention (PAA-WS₂, PAA-WSe₂, and PAA-MoSe₂ from the left, and a scale bar is 20 nm).

Referring to FIG. 1, the PAA-TMD nanosheets are well dispersed in a sheet form.

FIG. 2 shows the results of analysis of side size and height profiles of PAA-TMD nanosheets (PAA-WS₂, PAA-WSe₂, and PAA-MoSe₂ from the left).

FIG. 3 is a Raman spectrum analysis result of PAA-TMD nanosheets, and FIG. 4 is a FT-IR analysis result.

Referring to FIG. 3, characteristic peak appears in each spectrum, and it can be seen that the PDMS is well dispersed in a sheet form. In addition, referring to FIG. 4, PAA was well introduced into three kinds of PAA-TMD nanosheets according to an embodiment of the present invention.

### Antimicrobial Activity Assay

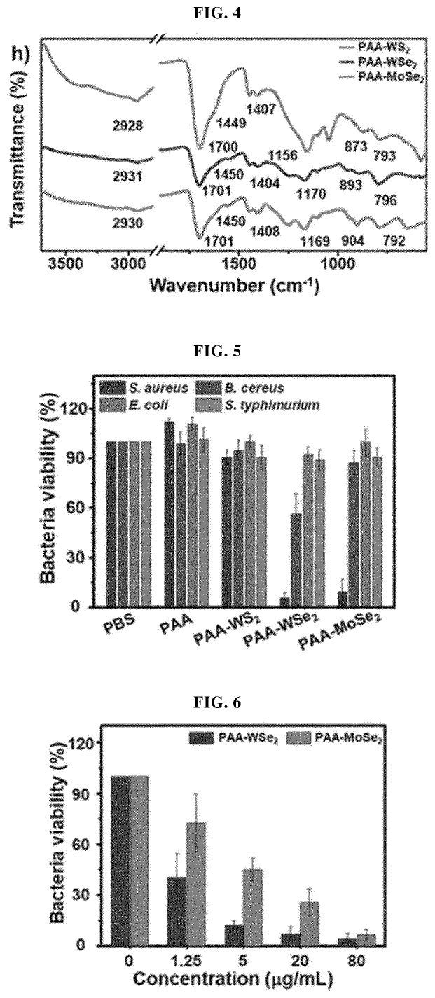

FIG. 5 shows the results of analysis of the relative survival rates of Staphylococcus aureus (S. aureus), Bacillus cereus (B. cereus), Escherichia coli (E. coli), and Salmonella typhimurium (S. typhimurium) treated with PAA-TMD nanosheets at a concentration of 20 g/mL.

Referring to FIG. 5, it can be seen that the Se-based TMD of the TDM has stronger antibacterial activity than the S-based TMD.

FIG. 6 shows the results of concentration-dependent antibacterial activity analysis of PAA-WSe₂ and PAA-MoSe₂ against Staphylococcus aureus (S. aureus).

Referring to FIG. 6, the survival rates of the S. aureus treated with PAA-WSe₂ and PAA-MoSe₂ at a concentration of 1.25 μg/mL were 40.5% and 72.5%, respectively, and the survival rates of the S. aureus treated with PAA-WSe2 and MoSe2 at a concentration of 80 g/mL were within about 5%.

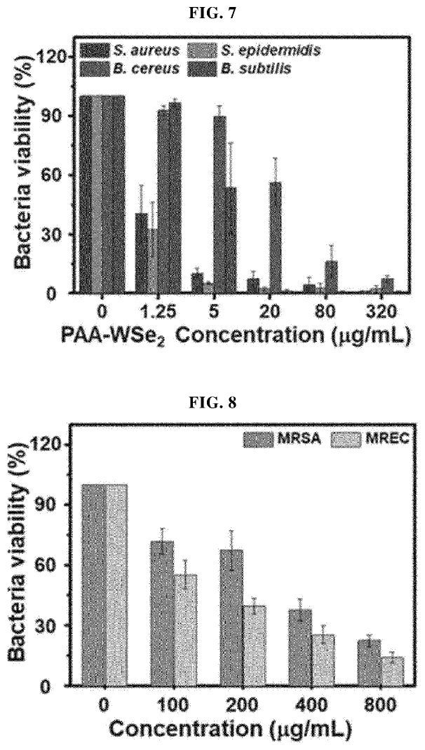

FIG. 7 is an analysis result of the antibacterial activity of PAA-WSe₂ against Gram-positive bacteria.

Referring to FIG. 7, the survival rate of Gram-positive bacteria treated with PAA-WSe2 at a concentration of 20 μg/mL was about 7% in S. aureus, about 2% in S. epidermidis, about 56% in B. cereus, and about 1% in B. subtilis.

FIG. 8 shows the results of analyzing the viability of multidrug-resistant Staphylococcus aureus (S. aureus) and Escherichia coli (E. coli) treated with PAA-WSe₂ or PAA-MoSe₂. In FIGS. 5 to 8, all error bars represent standard deviations of an average value (n=3).

Referring to FIG. 8, the survival rate of multi-drug resistant S. aureus treated with PAA-WSe₂ at a concentration of 800 μg/mL was about 22%, and the survival rate of multi-drug resistant E. coli was about 14%.

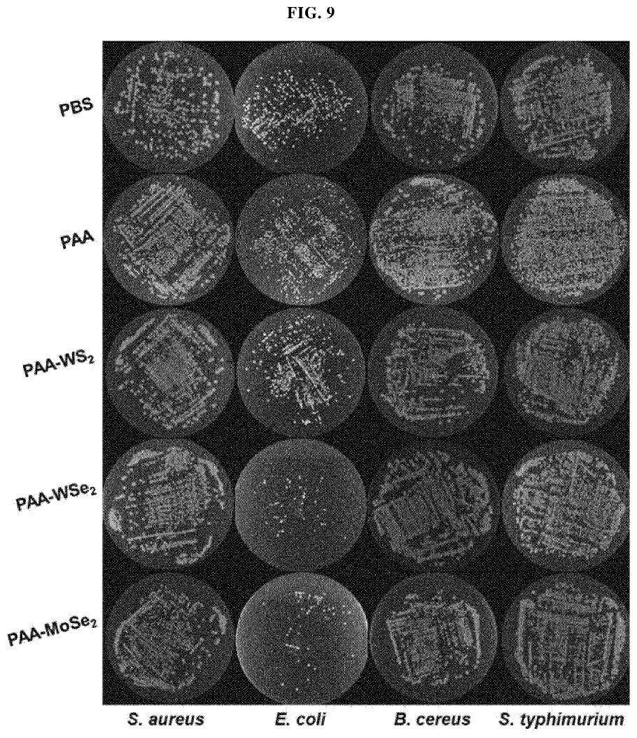

FIG. 9 is a picture of Staphylococcus aureus (S. aureus), Escherichia coli (E. coli), Bacillus cereus (B. cereus) and Salmonella typhimurium (S. typhimurium) colonies grown on nutrient agar plates after treatment with various concentrations of PAA or PAA-TMD nanosheets for 6 hours, in which the bacteria treated with each substance were raised on agar medium plates to observe survival rates

Referring to FIG. 9, no antibacterial activity was observed in the PBS and PAA treated groups, the antibacterial activity of PAA-WS₂ was observed to be weak, and the antibacterial activities of PAA-WSe2 and PAA-MoSe₂ were observed to be very strong.

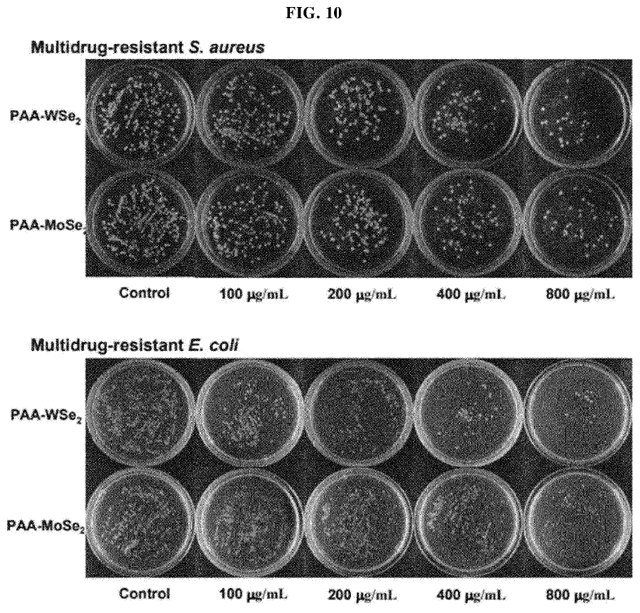

FIG. 10 shows pictures of multidrug-resistant Staphylococcus aureus (S. aureus) and Escherichia coli (E. coli) colonies grown on nutrient agar plates after treatment with various concentrations of PAA-TMD nanosheets, in which multidrug-resistant S. aureus, multidrug-resistant E. coli treated with PAA-WSe₂ and PAA-MoSe₂ were grown on agar medium plates and observed.

Referring to FIG. 10, it was confirmed that the survival rate was reduced depending on the concentration of the TMD nanosheet according to the present invention, thereby exhibiting antibacterial activity effective against multi-drug resistant bacteria.

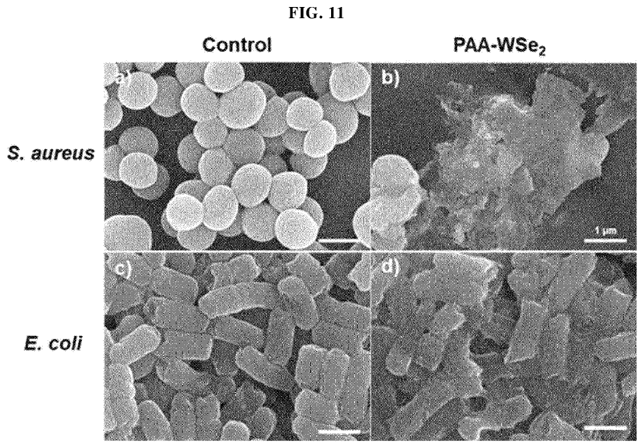

FIG. 11 is a scanning electron microscope (SEM) image of a)-b) Staphylococcus aureus (S. aureus), c)-d) Escherichia coli (E. coli), and shows results before and after treatment with PAA-WSe₂ (20 μg/mL) for 3 hours (scale bar: 1 m).

Referring to FIG. 11, it can be seen that S. aureus treated with PAA-WSe₂ was mostly destroyed within 3 hours, showing strong antibacterial activity, and E. coli maintained a slight appearance, showing relatively weak antibacterial activity.

1. An antibacterial composition comprising transition metal dichalcogenide (TMD) as an active ingredient.

2. The antibacterial composition of claim 1, wherein the transition metal dichalcogenide (TMD) is a nanosheet.

3. The antibacterial composition of claim 1, wherein the transition metal dichalcogenide comprises at least one selected from the group consisting of WS₂, MoS₂, WSe₂, and MoSe₂.

4. The antibacterial composition of claim 2, wherein the transition metal dichalcogenide (TMD) nanosheet is polyacrylic acid (PAA) functionalized by the transition metal dichalcogenide.

5. The antibacterial composition according to claim 4, wherein the dichalcogenide of the transition metal dichalcogenide (TMD) is Se.

6. A method for preparing the antibacterial composition of one claim 1, the method comprising: adding bulk TMD to a polyacrylic acid (PAA) solution to prepare a mixed solution; ultrasonically treating the mixed solution; centrifuging the ultrasonically treated mixed solution to obtain a precipitate from a supernatant; and redispersing the precipitate in water and centrifuging the same to obtain a PAA-TMD nanosheet from the supernatant.

7. The method for preparing the antibacterial composition of claim 6, wherein the transition metal dichalcogenide comprises at least one selected from the group consisting of WS₂, MoS₂, WSe₂, and MoSe₂.

8. A method for preparing the antibacterial composition of claim 2, the method comprising: adding bulk TMD to a polyacrylic acid (PAA) solution to prepare a mixed solution; ultrasonically treating the mixed solution; centrifuging the ultrasonically treated mixed solution to obtain a precipitate from a supernatant; and redispersing the precipitate in water and centrifuging the same to obtain a PAA-TMD nanosheet from the supernatant.

9. The method for preparing the antibacterial composition of claim 8, wherein the transition metal dichalcogenide comprises at least one selected from the group consisting of WS2, MoS2, WSe2, and MoSe2.

10. A method for preparing the antibacterial composition of claim 3, the method comprising: adding bulk TMD to a polyacrylic acid (PAA) solution to prepare a mixed solution; ultrasonically treating the mixed solution; centrifuging the ultrasonically treated mixed solution to obtain a precipitate from a supernatant; and redispersing the precipitate in water and centrifuging the same to obtain a PAA-TMD nanosheet from the supernatant.

11. The method for preparing the antibacterial composition of claim 10, wherein the transition metal dichalcogenide comprises at least one selected from the group consisting of WS2, MoS2, WSe2, and MoSe2.

12. A method for preparing the antibacterial composition of claim 4, the method comprising: adding bulk TMD to a polyacrylic acid (PAA) solution to prepare a mixed solution; ultrasonically treating the mixed solution; centrifuging the ultrasonically treated mixed solution to obtain a precipitate from a supernatant; and redispersing the precipitate in water and centrifuging the same to obtain a PAA-TMD nanosheet from the supernatant.

13. The method for preparing the antibacterial composition of claim 12, wherein the transition metal dichalcogenide comprises at least one selected from the group consisting of WS2, MoS2, WSe2, and MoSe2.

14. A method for preparing the antibacterial composition of claim 5, the method comprising: adding bulk TMD to a polyacrylic acid (PAA) solution to prepare a mixed solution; ultrasonically treating the mixed solution; centrifuging the ultrasonically treated mixed solution to obtain a precipitate from a supernatant; and redispersing the precipitate in water and centrifuging the same to obtain a PAA-TMD nanosheet from the supernatant.

15. The method for preparing the antibacterial composition of claim 14, wherein the transition metal dichalcogenide comprises at least one selected from the group consisting of WS2, MoS2, WSe2, and MoSe2.
